(19) United States
(12) Patent Application Publication
(10) Pub. No.: US 2018/0015387 A1
(43) Pub. Date: Jan. 18, 2018

(54) SYSTEM AND METHOD OF FORMING A TERPENE SOLUTION

(71) Applicant: Connoisseur Holdings, LLC, Tigard, OR (US)

(72) Inventor: Andrew Jones, Tigard, OR (US)

(21) Appl. No.: 15/650,754

(22) Filed: Jul. 14, 2017

Related U.S. Application Data
(60) Provisional application No. 62/363,249, filed on Jul. 16, 2016.

(51) Int. Cl. B01D 11/04; C11B 3/00; A61K 36/18

(52) U.S. Cl. CPC B01D 11/04; A61K 36/18; C11B 3/006

(57) ABSTRACT

The disclosed system and method of forming a solution for use with a base to form a product includes identifying various characteristics of the base the solution is to be used with, various product characteristics, including the use of the product and the device employed to use the product. Additionally, the solution can be formed based on one or more user experience characteristics of the base, to optimize a user's experience with the resultant product while considering the characteristics and/or properties of the base and the product.

### CROSS-REFERENCE TO RELATED APPLICATIONS

This application claims the benefit of Provisional Patent Application 62/363,249, filed Jul. 16, 2016, the contents of which are all incorporated herein in their entirety.

### BACKGROUND

People have been consuming and/or using plant extracts for a variety of purposes throughout humanity. From medicinal to recreational purposes, plant extracts have provided many benefits. Depending on the plant and the extract, the plant extract can take many forms, from a liquid to a solid material. The diverse chemical compositions and forms of the extracts can make consumption and/or use of the plant extracts difficult for a user and/or consumer.

The use plant extracts and the various methods that humans employ to use, consume, and manipulate plant based extractions is a growing field. As the use and consumption of plant extracts has grown, there has emerged the necessity to create solutions that can prepare the plant extracts for safe and pleasurable use and consumption. There exists a need for a system and/or method that can be used to construct and/or formulate a solution for use with a base, such as a plant extract, that results in a product having known and/or desired properties and characteristics depending on the use of the product.

### BRIEF DESCRIPTION OF THE DRAWINGS

FIG. 1 is a block diagram of an example product.

FIG. 2 is a block diagram of an example solution.

FIG. 3 is an example process for the creation of a solution.

### DETAILED DESCRIPTION

The disclosed methods and systems of making a terpene solution for use with a base, such as a plant extract, form a resultant product when the base and solution are combined, the product having one or more desirable characteristics. The desirable characteristics can include optimizing the product for consumption by a user, such as improving the flavor and/or usability of the product. The solution is formed based on characteristics and/or properties of the base it is to be mixed with and the desired product characteristics, such as a device the product will be used with, how the product will be used, user experience characteristics and/or other considerations. The characteristics of the base can be enhanced, reduced, added to, and/or negated by the solution, such that the combination of the solution and base results in a product having at least one desired, preferred, or otherwise different characteristic than that of the base.

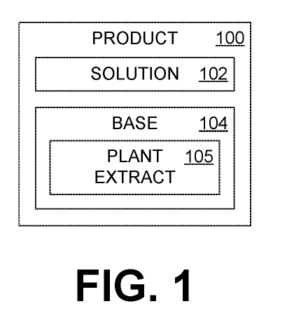

FIG. 1 is a block diagram of an example product 100 formed by the combination of at least the solution 102 and a base 104. The product can have desirable or preferred characteristics and/or properties that are caused by the mixing of the solution 102 with the base 104. One or more of the various properties and/or characteristics of the product 100 can be the same as that of the base 104, a solution 102 modified characteristic or property of the base 104, or a property or characteristic of the solution 102.

The solution 102 can be constructed and/or formulated based on one or more of the desired or preferred properties and/or characteristics of the product 100, one or more of the properties and/or characteristics of the base 104, and/or various other considerations. The constructed and/or formulated solution 102 can contain various materials, compounds and/or elements selected in various measures to create a product 100 having at least a desired, preferred or known characteristic or property when the solution 102 and base 104 are combined.

To construct and/or formulate the solution, various product 100 properties and/or characteristics can be determined, and/or the base 104 can be analyzed to determine various properties and/or characteristics of the base 104. Based at least in part on one or both the product 100 and base 104 properties and/or characteristics, the solution 102 can be constructed and/or formulated.

Determining the various desirable and/or preferable properties and/or characteristics of the product 100 can be based in part on a method and/or a device by which the product will used and other various considerations, such as a flavor, aesthetic and/or other considerations of the product 100. The solution 102 can be constructed and/or formulated to increase, decrease, maintain, or substantially negate an existing property or characteristic of the base 104 or add preferred property or characteristic, such that when the base 104 and solution 102 are combined to form the product 100, the product 100 includes the desired or preferred property and/or characteristic, or value thereof.

In an example, the product 100 might be required or desired to have a property and/or characteristic, or an amount thereof, that allows the product 100 to be used with a particular device. The base 104 might lack that particular property and/or characteristic or might have an amount or measurable magnitude different from a required/desired amount or outside of a required/desired range. The solution 102 can be constructed and/or formulated such that when the solution 102 and base 104 are combined, the resultant product has the particular property or characteristic, or the required or desired magnitude, or is within the suitable magnitude range, of the particular property or characteristic.

Determining the various properties and/or characteristics of the base 104, which can assist in the construction and/or formulation of the solution 102, can be done using a variety of methods, device and means. Various analyses of the base 104 can be carried out, such as spectral analysis, chromatography, and others, to determine the composition, relative concentrations of components of the base 104, and other structural and/or chemical properties and/or characteristics of the base 104. Additionally, the analysis of the base 104 can determine a chemical profile, such as a “chemical signature,” of the base 104. The chemical profile can list various compounds and concentration of various compounds within the base 104. The solution 102 can be constructed and/or formulated compete with and/or compliment one more of the elements of the chemical profile.

The analysis of the base 104 can also be performed in light of the desired and/or preferred property and/or characteristic of the product 100. For example, the product can be desired or required to have a certain viscosity, the viscosity of the base 104 can therefore be determined to determine an adjustment of the base 104 viscosity by the solution 102 that is necessary to achieve the required and/or desired viscosity of the product 100.

By knowing at least one of the desired and/or preferred product 100 properties and/or characteristics and various properties and/or characteristics of the base 104, the solution 102 can be constructed and/or formulated to augment and/or reduce one or more properties and/or characteristics of the base 102 and/or add a property and/or characteristic to achieve the desired or preferred product 100 properties and/or characteristics.

The base 104 can be various materials, compounds and/or combinations thereof. The base 104 can be naturally derived, synthetically created and/or a combination thereof. The various constituents, compounds and/or elements that comprise the base 104 can be organic, inorganic or a combination thereof. An example base 104 can include a plant extract 105. The plant extract 105 can be one or more of a variety of plant derived extracts and/or compounds that a user may wish to utilize and/or consume. An example plant extract 105 can include a cannabis based extract, which can include one or more cannabinoids in various concentrations. The solution 102 can be constructed and/or formulated to assist with the preparation, utilization and/or consumption of the cannabis extract base by a user. The solution 102 can be further constructed and/or formulated to enhance and/or reduce various aspects of consumption of the cannabis extract, such as effecting various user experience properties of the cannabis extract, which could include a psychoactive effect of the cannabis extract.

In the example cannabis extract, the extract can include a natural component that is derived, extracted, refined, or otherwise obtained from a natural source, such as the cannabis plant, a synthetic component that is manufactured, such as to mimic or be substantially similar to a component of the natural source, or a combination thereof. That is, the base 104 cannabis extract can include or be composed of natural components, synthetic components, or a combination thereof. For example, the base 104 can include a synthetic cannabinoid that is structured to mimic or induce similar effects in a user as a natural cannabinoid, or can include combination of a naturally derived component(s) and a synthetic component(s) to mimic or mirror a cannabis extract.

The solution 102 can be delivered, purchased, and/or used in a kit, which can include the solution 200 and/or various components of the solution 102. An example kit can include the components of the solution 102 themselves, alone or in combination with other components; a deliverable substance or base 104, such as a drug, a plant extract, a pharmaceutical ingredient; a delivery, or use, device, and/or a mixing structure, such as a vial or other suitable sized receptacle. The various solution 102 components of the kit and the base 104 can come individually, requiring a user to mix the components as directed or desired to form the product 100, come premixed as the product 100, or some combination or combinations thereof. The delivery device of the kit can mix the various components of the solution 102 in pre-determined amounts, as desired by the user or a combination of the two, allowing a user to modify the pre-determined amounts to customize the resultant solution 102. The delivery device can also mix the solution 102 and the base 104 in a selected, required and/or desired ratio to deliver the product 100 to the user. In an example embodiment, the delivery, or use, device of the kit can come with at least a portion of the solution 102 components installed within the delivery device by a manufacturer or provider of the kit. Alternatively, a solution 102 to be used with the delivery, or use, device can be created or mixed by the user and inserted into the delivery, or use, device for ingestion or consumption by a user or other. In another embodiment, the kit may require the user supply at least a missing portion of the kit, such as the base, to create and/or deliver the final product 100.

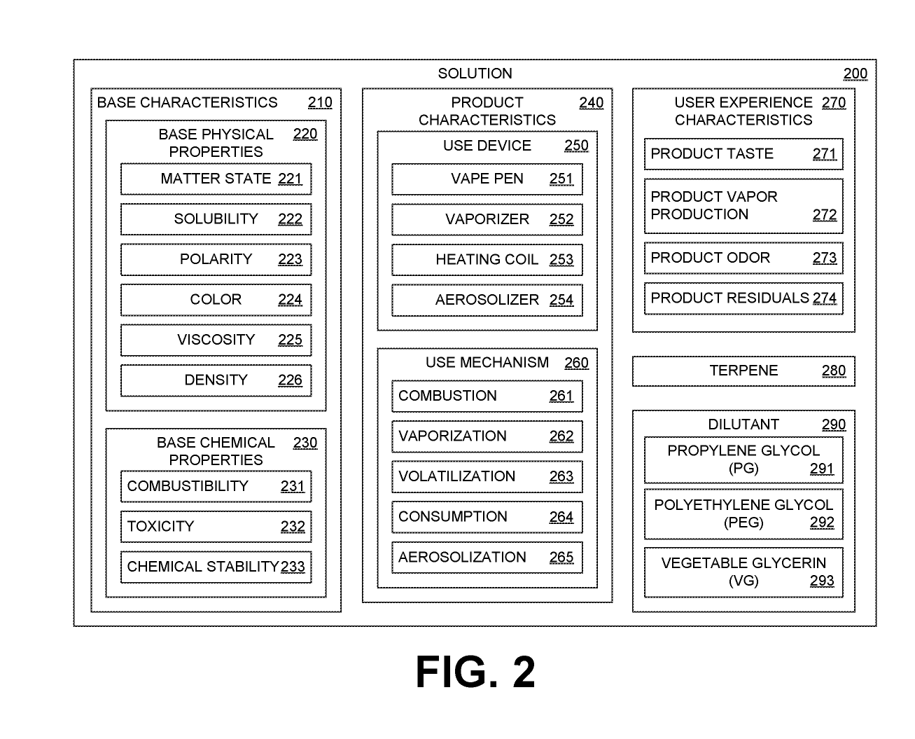

FIG. 2 illustrates an example solution 200 that is constructed, or formulated, based at least in part on base characteristics 210, product characteristics 240 and/or user experience characteristics 270. The solution 200 can include various terpenes 280 and/or dilutants 290 that can be selected and/or mixed to construct the solution 200, or portion thereof. The solution 200 can be mixed with a base, such as a plant extract, to form a product that can then be used in various method and/or devices for consumption by a user.

The solution 200 can be constructed and/or formulated based on various base characteristics 210 which can include various base physical properties 220 and/or base chemical properties 230. The base characteristics 210 are properties and/or characteristics of a base, such as a plant extract. When the base is combined with the solution 200, the resulting product has overall properties and/or characteristics that are partially a combination of the base characteristics 210 and the properties and/or characteristics of the solution 200. The resulting product can have desirable and/or preferable properties and/or characteristics for consumption of the product by a user.

The various base characteristics 210 can be known characteristics of the base and/or can be determined using various analytical techniques. Example analysis of the base to determine base characteristics 210 can include spectral analysis, gas chromatography, and other analysis techniques to determine structural and other various properties and/or characteristics of the base.

The base physical properties 220 can be various physical properties of the base, such as a plant extract, and can include a matter state 221 of the base, solubility 222 of the base, polarity 223 of the base, color of the base 224, viscosity 225 of the base and/or density 226 of the base. The solution 200 can be formulated based in-part on these and/or other physical properties 220 of the base, such that the resultant combination of the solution 200 and base creates a product having at least a known and/or desirable property and/or characteristic.

The mater state 221 of the base can include whether the base is a solid, a liquid or a gas, including the likelihood of the base to transition to a second state of matter different from the initial state of matter of the base. For example, if the base is in a gaseous state, the solution can be constructed and/or formulated to capture the gaseous base so that the base can be consumed or otherwise used by a user. In a further example, the base can be a solid and the preferable product can be a liquid, the solution can dissolve and/or otherwise suspend the base in a solution to result in the preferably liquid product. In yet a further example, the solution can assist with maintaining the matter state of the base, such as preventing the transition of a solid-state base into a liquid or preventing a liquid base from transitioning to a gaseous state, or other various matter state transitions.

The solubility 222 of the base is the characteristic of the base to dissolve in a solvent, such as the solution 200. The solubility 222 can also include the suspension of the base in the resulting product and the duration the base stays dissolved in, or in solution with, the product before precipitating out or separating from the solution 200. The solution 200 can be constructed and/or formulated to and/or assist with the dissolving and/or suspension in solution of the base and maintaining the base dissolved and/or suspended in the resulting product. The ability of the solution 200 to dissolve the base and maintain the base in a dissolved state can determine the stability of the resulting product when the base and solution 200 are combined. A more stable product can extend the usable life of the product which can limit wastage by allowing a user suitable time to consume or use the product.

Polarity 223 of the base is the distribution of charge on the molecules of the base. The molecules of the base can have a localized positive and/or negative charge of the molecule which can affect the ability of the base to be mixed with other materials. The solution 200 can be constructed and/or formulated based on the polarity of the base to assist with the suspension and/or dissolving of the base with/in the solution 200 to form a resulting product 200.

The solution 200 can also be constructed and/or formulated based on the color 224, or lack thereof, of the base to result in a product having a desired and/or preferable color, or lack thereof. The color of the resultant product, formed by the combination of the solution 200 and the base, can be used to indicate various information to a consumer, such as a flavor of the product or branding the product. Additionally, the solution 200 can be constructed and/or formulated to result in a product having a color indicative of a base, such as a type of base, i.e. a specific plant based extract, the purity or strength of the base and/or other various properties and/or characteristics of the base.

The viscosity 225 of the base is a measure of the “thickness,” or the ability to flow, of the base. The solution 200 can be constructed and/or formulated such that when the solution 200 and base are combined, the resulting product has a desired and/or preferable viscosity. The desired and/or preferable viscosity of the product can be based on the method and/or device to be employed by a user to consume the product. An example base can be a solid that when combined with the solution 200 to result in a product having a desired and/or preferable viscosity. The desired, preferred and/or required viscosity of the resultant product by combining at least the solution 200 and the base can be based on the use device and/or use mechanism the product will be used with. That is, a certain device a user employees to use and/or consume the product might require a product of a certain viscosity, or range of viscosities, and/or may work optimally with a product having a certain viscosity, or range of viscosities. The solution 200 can be constructed and/or formulated such that the resultant product has the required, desired, and/or preferred viscosity for use with a particular use device and/or use mechanism.

Along with the viscosity 225, the base can also have a density 226, a mass per unit volume. As with the other base physical properties 220, the solution 200 can be constructed and/or formulated to result in a product having desired properties and/or characteristics, such as a product density.

Another of the base characteristics 210 can include base chemical properties 230. The base chemical properties 230 can include a combustibility 231 of the base, a toxicity 232 of the base, a chemical stability 233 of the base and/or other various chemical properties 230. Various chemical properties 230 of the base can determine the chemical interaction between the base and the solution 200. The solution 200 can be constructed and/or formulated based on the base chemical properties 230 such that when the solution 200 and base are mixed, the resultant product has desired and/or preferred properties and/or characteristics.

Combustibility 231 is the property and/or characteristic of the base to catch fire and/or combust. The solution 200 can be constructed and/or formulated such that the resulting product formed by mixing the base and solution 200 has a desired and/or preferred combustibility. The combustibility of the resultant product can be engineered for use with a desired method and/or device of consumption of the product by the user.

The base can also have a toxicity 232 that can affect the health of the consumer who uses the product. The solution 200 can be constructed and/or formulated to reduce and/or negate the toxicity 232 of the base when the base and solution 200 are mixed to form a resultant product. The solution 200 can contain various compounds and or chemicals that can interact with the base to and/or the solution 200 can dilute the base to reduce and/or negate the toxicity 232 of the base.

The chemical stability 233 of the base is the ability of the base to remain chemically unaltered over time. The solution 200 can be constructed and/or formulated based on the chemical stability 233 of the base to create a resultant product having a desired or preferred chemical stability. The chemical stability of the product can determine the length of time the product, a combination of the solution 200 and the base, can remain stable and usable.

The solution 200 can be constructed and/or formulated based on various product characteristics 240, including a user device 250, a use mechanism 260 and/or other various product characteristics 240. The product characteristics 240 can be various use methods and device of the resultant product formed by the combination of the base and the solution 200.

The use device 250 can include various devices a user can employ to consume the product of the solution 200 and the base. Many various use devices 250 can be used by the user and each of the various use devices 250 can include various elements, components and/or systems to deliver the product and/or assist the user with utilizing and/or consuming the resultant product. The use devices 250 can use one or more use of the use mechanisms 260 or other delivery methods, systems and/or components. Example use devices 250 can include a vape pen 251, a vaporizer 252, heating coil 253, an aerosolizer 254 and/or other devices a user can use to utilize and/or consume the product resulting from the combination of the solution 200 and a base.

The vape pen 251 is a self-contained device that a user or another can insert the solution 200 and a base, that the vape pen 251 can then process to form and/or deliver a product of the base and solution 200, such as by aerosolizing the product by combustion 261, an example product use mechanism 260. Alternatively, the user or another can insert a product of the solution 200 and a base that the vape pen 251 can then process to deliver, such as by aerosolizing the product. The product to be vaporized is heated by a heating, or other, element of the vape pen 251 to combust 261 the product into an aerosolized form. The product, formed by the solution 200 and a base, can be required to have, or can preferably have, one or more properties and/or characteristics. The solution 200 can be constructed and/or formulated such that the resultant product formed by the combination of at least the base and the solution 200 has the one or more properties and/or characteristics. The one or more required properties and/or characteristics of the product to be used with the vape pen 251 can include those that optimize the product for use with the vape pen 251, including the safe and efficient operation of the vape pen 251 and delivery and/or use of the product by the user.

In an example, it may be required that the product have a minimum viscosity for use in the vape pen 251 and the product can further have an ideal range of viscosities for improved and/or optimal use of the product by the vape pen 251. The solution 200 can be constructed and/or formulated to result in a product, when the solution 200 and base are combined, having a viscosity greater than the minimum required and preferably within the ideal range of viscosities. The viscosity of the product as used by the vape pen 251 can assist with the performance of the vape pen 251, such as by preventing or minimizing leakage of the product from the vape pen 251 when not in use, and optimizing the performance of the vape pen 251 and its delivery of the product.

Another consideration of constructing and/or formulating the solution 200 for use with a vape pen 251 can include the heat of combustion of the resultant product when the solution 200 and the base are combined. The solution 200 can be constructed and/or formulated so that the resultant product has a heat of combustion suitable for use with the vape pen 251. Various vape pens 251 can have varying amounts of heat they can generate to combust the product. Various solutions 200, or various solution additives, can be constructed and/or formulated to combine with the base depending on the heat generation capability, or an ideal, efficient, and/or safe heat generation capability, of a particular vape pen 251. A solution 200 tailored, or solution additive(s) added to tailor the solution 200, to result in a product having a particular heat of combustion can increase the safety of using a particular vape pen 251 since the heating element of the vape pen 251 can safely operate within a normal operating range, thus limiting the potential of the heating element to overheat and result in a potentially user hazardous situation.

A vaporizer 252, or a vaporization device, is further use device 250 that can be employed to use and/or consume the product formed by the combination of at least the base and the solution 200. Vaporizers 252, typically use conduction and/or convection as the methods of heating a product, such as one created by the combination of at least the base and the solution 200. Conduction is the direct application of heat to the product to heat and vaporize at least a portion of the product. Convection is the indirect application of heat to the product by movement of a heated medium, such as air, about the product to heat and vaporize at least a portion of the product. Various components and/or compounds of the product are volatilized at different temperatures. By controlling the temperature of the vaporization device, desired and/or selected components and/or compounds of the product can be vaporized for use and/or consumption by a user.

Vaporizers 252 that utilize a conductive method of heating can include a surface, such as a solid metal surface or metal screen, that can be heated. The product can be placed upon this surface and in turn be heated, or be placed on the already heated surface, to vaporize one or more components and/or compounds of the product.

Vaporizers 253 that utilize convective heating keep the product to be vaporized from directly contacting the heated surface. The device can use heating elements to heat air to a desired temperature, that heated air can then be passed about the product, usually through the assistance of a fan or inhalation of air through the device by a user, causing heat from the heated air to be transferred to the product. One or more components and/or compounds of the product can then be volatilized due to the heating of the product.

The solution 200 can be constructed and/or formulated to create a resultant product, when combined with the base, that has required, desired and/or preferred characteristics and/or properties for use with a vaporizer 253. The required, desired, and/or preferred characteristics and/or properties of a product for use with a vaporizer 252 can be related to the type of vaporizer, such as whether the vaporizer 252 uses a conductive or convective heating method. Additional considerations, such as the temperature, or temperature range, at which one or more desired compounds and/or components of the product vaporize, can be considered when constructing and/or formulating the solution 200 for combination with at least the base to create the product to be vaporized. In an example, the solution 200 can be constructed and/or formulated to result in the product having an increase or reduced temperature, or temperature range, of vaporization, which can align with an operating and/or optimal temperature, or temperature range, of the vaporizer 252.

A heating coil 253 is a further use device 250 which can be included in the product characteristics 240 upon which the constructed and/or formulated solution 200 can be based. The heating coil 253 can include a heating element and/or a heatable surface upon which the product, formed by the combination of at least the solution 200 and the base, can be placed. Example heating coils 253 can include an aromatherapy device and a ceramic coil that can be heated. The ceramic coil is porous allowing a liquid product to be applied to and absorbed by the ceramic coil. As the ceramic coil is heated, the product, or components and/or compounds thereof, can be released and used and/or consumed by a user. The product can be volatilized, vaporized, combusted and/or otherwise released due to the heating of the heating coil 253 upon which the product is placed. Alternatively, the solution 200 and the base can be separately placed on the heating coil 253, or on/in another use device 250, and can combine on the heating coil 253 to result in a product having at least a required, desired, and/or preferred characteristic and/or property. Further example heating coils 253, can include a lightbulb or a resistive heating element, which can include a cover or other surface, upon which the product, and/or solution 200 and base, can be placed to assist with the use and/or consumption of the product.

A further use device 250 can include an aerosolizer 254 that can assist a user with the use and/or consumption of the product formed by the combination of at least the solution 200 and the base. The aerosolizer can use a mechanical system and/or device to aerosolize the product, including passing the product through a nozzle or vibrating the product using an ultrasonic vibrator. The product can be placed in/on the aerosolizer 254, and/or the solution 200 and base can be placed separately in/on the aerosolizer 254 and combined by the device to form the product, for aerosolizing. An aerosol of the product can include a mist of or small droplets of the product that can then be used and/or consumed by a user. The solution 200 can be constructed and/or formulated to create a resultant product, when the solution 200 is combined with the base that has properties and/or characteristics that can assist with the aerosolization of the product. Example properties and/or characteristics that can assist with aerosolization of the product can include a desired viscosity or viscosity range of the product, the miscibility of the base and solution 200 and droplet/aerosol formation characteristics of the product.

The product characteristics 240, upon which the construction and/or formulation of the solution 200 can be based, can include one or more use mechanisms 260. The use mechanisms 260 are various mechanisms by which the product, the combination of at least the solution 200 and the base, can then be ultimately provided to a user for use and/or consumption. As previously mentioned, the use devices 250 can utilize one or more of the use mechanisms 260 to assist with the delivery of the product to a user, or can utilize other use mechanisms 260. Example use mechanisms 260 can include combustion 261, vaporization 262, volatilization 263, consumption 264, aerosolization 265 and/or other use mechanisms 260.

The use mechanism 260 can also release of a controlled or measured amount of the product, and/or components/compounds of the product, for use and/or consumption by the user. The solution 200 can be constructed and/or formulated so that when the solution 200 and base are combined to form a product, the product has a known concentration, or concentration range, of a particular compound and/or component that will be released by the use mechanism 260 to the user. Using the concentration of the particular compound and/or component of the product, a known amount, such as a dose, of the particular compound and/or component can be released by the use mechanism 260. Other methods of facilitating a controlled or measured release amount of a particular compound and/or component of the based and/or product can be employed utilizing a use mechanism 260.

Combustion 261 is a process by which the product, created by the combination of at least the solution 200 and the base, is burned, or combusted. The resultant vapor created by the combustion 261 of the product can include one or more components and/or compounds, which can be altered due to the combustion 261 process, of the product. The solution 200 can be constructed and/or formulated with consideration to a combustion 261 use mechanism 260. For example, the solution 200 can be constructed and/or formulated such that the resultant product has a desired combustion temperature or temperature range that aligns or is based on the temperature of the combustion 261 use mechanism 260. The solution 200 can also be constructed and/or formulated based on other consideration of utilizing combustion 261 as a use mechanism 260 of the product formed by the combination of at least a base and the solution 200.

Vaporization 262 is a further use mechanism 260 by which a product, formed by the combination of at least the solution 200 and a base, can be utilized and/or consumed by a user. Vaporization 262 is a process by which a substance transitions from a liquid state to a gaseous state. The vaporization 262 of a product can include vaporization of the product, or a portion, such as a particular compound and/or component, thereof. Vaporization 262 of the product can include heating the product to cause the product, or a component/compound of the product, to vaporize. The vapor formed by vaporizing 262 the product, or a portion thereof, can be used and/or consumed by the user. The solution 200 can be constructed and/or formulated to assist with, facilitate and/or otherwise result in a product, formed by at least a base and the solution 200, that can be vaporized 262, or a portion of the product thereof. For example, the solution 200 can be constructed and/or formulated to create a resultant product, or a portion thereof, when combined with a base that has a required, desired, and/or preferred vaporization characteristics and/or properties, such as a heat of vaporization of the product or a portion thereof.

Volatilization 263 is a further use mechanism 260 that can be utilized by a user to use and/or consume a product, formed by the combination of at least the solution 200 and a base, or a portion thereof. Volatilization 263 is similar to vaporization 262 in that a product, or a portion thereof, is converted into a gaseous state or a vapor. Unlike vaporization 262, volatilization typically a passive process. Rather than adding heat energy to the product to vaporize the product, or a portion thereof, volatilization can use the ambient conditions to facilitate the evaporation, or vapor formation, of the product or a portion thereof. The solution 200 can be constructed and/or formulated at least in part on volatilization 263 being a use mechanism 260 of the product. The resultant product formed by combining at least the solution 200 and a base can have required, desired and/or preferred volatilization 263 properties and/or characteristics. For example, the solution 200 can result in a product, or a portion thereof, having a volatilization temperature and/or temperature range. In an example in which the solution 200 is constructed and/or formulated to interact with a particular compound/component of the base such that the particular compound/component has a known volatilization temperature, in this manner, exposure of the product to a suitable temperature will cause volatilization of the particular compound/component. Various other product volatilization properties and/or characteristics can be considered and used to construct and/or formulate a solution 200, that when combined with a base, results in a product having required, desired and/or preferred volatilization properties and/or characteristics.

Consumption 264 is a further use mechanism 260 that can be utilized by a user to use and/or consume a product. Consumption 264 can include the direct consumption of a product by a user, such as through ingestions, injection, inhalation, absorption, and/or other means of introducing the product directly to the consumer. The solution 200 can be constructed and/or formulated such that the resultant product, formed by combining at least the solution 200 and a base, has required, desired and/or preferred characteristics and/or properties based on consumption 264 being a use mechanism 260 of the product. For example, the solution 200 can be constructed and/or formulated such that the product has a desired flavor or to make the resultant product safe for consumption by a user. Various consideration and aspects of consumption 264 of the product can be used to base the construction and/or formulation of the solution 200 upon.

A further use mechanism 260 is aerosolization 265. Aerosolization 265 of the product can include creating a mist and/or small droplets of the product. The aerosolized product can then be used and/or consumed by the user. The solution 200 can be constructed and/or formulated based on the product, formed by the combination of the solution 200 and a base, use mechanism 260 being aerosolization 265. For example, the solution 200 can be constructed and/or formulated such that the resultant product has a low viscosity enabling the product to be more easily aerosolized 265 than a thicker, more viscous product. Additional considerations, such as the agglomeration characteristics/properties of the product can be considered to assist with forming an aerosol of product having required and/or desired droplet size. The solution 200 can be constructed and/or formulated based on various considerations of the product use mechanism 260 to be utilized, aerosolization 265 or other use mechanisms.

The solution 200 can also be constructed and/or formulated based on various user experience characteristics 270. User experience characteristics 270 can be a variety of sensations, thoughts, feelings, and other user experience's that a user can experience while using the product formed by the combination of at least the solution 200 and a base. The user experience characteristics 270 can be subjective or objective measures of a user's satisfaction and/or other experience's from using the product. The user experiences 270 can include a product taste 271, a product vapor production 272, a product odor 273, product residuals 274 and/or other user experience characteristics 270. The solution 200 can be constructed and/or formulated to create a resultant product, when combined with a base that has required, desired and/or preferred user experience characteristics 270 and/or measures the user experience characteristics 270.

The product taste 271 user experience characteristic 270 can include the taste or flavor of the product the user experiences during and/or after use of the product. A user can desire a certain product taste 271 and the solution 200 can be constructed and/or formulated such that when the solution 200 and a base are combined, the resultant product has the desired product taste 271. In an example, a base can have an undesirable or less desirable flavor that the constructed and/or formulated solution 200 can be combined with the base to result in a product having a more palatable flavor to the user. The solution 200 can be constructed and/or formulated to enhance or mask a flavor, or aspect thereof, of a base to create a product having a desired and/or preferred product taste 271. For example, a base can have desirable and/or preferred flavor notes, or aspects of the flavor of the base, that the solution 200 can accentuate in the resultant product. Alternatively, the base can have undesirable or less desirable flavor notes that the solution 200 can mask or reduce in the resultant product. That is, the base can have a flavor profile, or portions thereof, that the solution can enhance, modify, reduce, mask or otherwise alter based on a desired and/or preferred user product taste 271.

The product vapor production 272 user experience characteristic 270 can include the amount and other characteristics/properties of the vapor formed by the use device(s) 250 and/or use mechanism(s) 260 a user utilizes to use and/or consume the resultant product formed by combing at least the solution 200 and a base. The solution 200 can be constructed and/or formulated such that the resultant product has a required, desired, and/or preferred vapor production 272. A user may prefer or desire a large vapor production 272, which may be perceived as associated with a high-quality of the product. The solution 200 can be constructed and/or formulated to deliver this desired user experience characteristic 270 associated with the product formed by at least the combination of the solution 200 and a base. Alternatively, the user may wish to be discrete in their use of the product, so a product with a lower vapor production 272 can be preferred. The solution 200 can be constructed and/or formulated to deliver this desired user experience characteristic 270 associated with the product formed by at least the combination of the solution 200 and a base. The product vapor production 272 can also vary depending upon the use device 250 and/or another product 240 or base characteristic 210. The solution 200 can be constructed and/or formulated based on one or more of the base 210 and product characteristics 240 to create a product, when the base and the solution 200 are combined, having the desired vapor production 272.

The product odor 273 user experience characteristic 270 can include the odor of the product during use, such as the odor of the formed vapor or other form the product is used and/or consumed by the user, the odor of the product itself and/or other product odor characteristics and/or properties. A user can desire the product have a low odor so as to be unobtrusive in use, a pleasing odor during use and/or other odor considerations. The solution 200 can be constructed and/or formulated based on the desired and/or preferred user product odor 273 such that the resultant product, of the combination of the solution 200 and a base, has the desired odor characteristics and/or properties. For example, the solution 200 can be constructed and/or formulated such that the product odor is pleasing to the user and others that may be nearby the user. Alternatively, the solution 200 can be constructed and/or formulated such that the product has little or no odor during use so the user's use of the product is unobtrusive or not easily detected by others.

Product residuals 274 can include the byproducts formed by or result from the use of the product in a use device 250 and/or by a use mechanism 260. For example, product residuals 274 can include uncombusted portions of the product and/or product combustion byproducts that remain after a user's use and/or consumption of the product. The product residuals 274 can require regular maintenance to remove and/or remedy to maintain the safe and/or efficient use of the use device 250 and/or use mechanism 260. For example, product residuals 274 can cause a premature failure or an unsafe operating condition of a use device 250, such as build up on the heated surface of a vape pen 251. The solution 200 can be constructed and/or formulated such that the resultant product, formed by the combination of at least the solution 200 and a base, minimizes and/or reduces the product residuals 274 from use of the product. For example, the solution 200 can include a combustible component that can assist with the substantially complete combustion of the product during use to minimize product residuals 274. The solution 200 can also include various use device 250 and/or use mechanism 260 cleaning components that can assist with reducing the formation and/or cleaning of the product residuals 274. These cleaning components can be selected to have minimal and/or no impact on other user experience characteristics 270.

The solution 200 can be constructed of, formulated of, and/or include a variety of compounds and/or components, including terpenes 280. Terpenes are a large and diverse class of organic compounds, which are produced by a variety of plants and insects. Many terpenes are similar to alcohol in appearance, and often have a strong scent. Another large and diverse class of organic compounds are terpenoids which are similar to terpenes. Terpenes are hydrocarbons, often based on units of isoprene (C₅H₈), while terpenoids contain additional functional groups and are generally thought of as modified terpenes. Individual terpenes or combinations of various terpenes can be mixed to form a terpene-based solution 200. The terpene-based solution 200 can be mixed with a base to form a liquid, terpene-based product. The base can be dissolved in, suspended in, or mixed with the terpene-based solution create a product having required, desired and/or preferred characteristics and/or properties.

Terpenes 280 can also be used to create a flavor of the resulting product. The terpene-based solution can be constructed, formulated, and/or designed to have various flavor profiles as desired. The terpene-based solution can be used to flavor a resultant product as desired. A variety of solutions 200, each having a flavor profile, can allow a user to choose or select a terpene-based, or other, solution that is pleasing to their palate and can be a marketable aspect of the various terpene-based, and other, solutions and terpene-based products.

The terpenes 280 can be derived from natural sources, such as plants, or can be synthetically created, such as in a laboratory or manufacturing facility. Some terpenes, due to their difficulty of manufacture, cost to manufacture, concentration in a natural source, rarity of the natural source and/or other factors, can have a relatively high cost. While the solution 200 formula and/or construction can call for a small amount of a particular terpene 280, a relatively high cost of that terpene 280 can make the solution therefore relatively high cost. An alternative terpene(s) 280 can be used in substitution or the relatively high cost terpene 280 can be used in the solution 200, dependent on a various manufacturer, consumer, market, and/or other considerations. For example, exotic or hard to extract/reproduce terpenes can be economically unfeasible for mass market or large volume extract solutions. α-pinene, a terpenoid, for example, easily extracted from pine needle has a very low cost, while α-amyrin can have a cost a thousand times more expensive than that of α-Pinene. Expensive or high cost dilutants can be used when necessary or desired to create unique or limited edition extract solutions.

In addition to other properties, many terpenes have been found to have medicinal benefits and can be administered in a variety of forms, including ingestion, inhalation, and aromatherapy. Pinene, a bicyclic monoterpenoid, can be used medicinally as an anti-inflammatory, an expectorant, a bronchodilator, and a local antiseptic. The potentially broad-reaching medicinal value of terpenes and terpenoids is not yet fully understood, but with the number of known terpenes reaching into the hundreds further research promises exciting new applications.

In addition to the individual benefits of terpenes, there is emerging scientific evidence of an entourage effect, where the combination of a terpene together with other terpenes or a base material, such as a plant material and/or extract, are multiplying or increasing the positive effects of the individual ingredients. The entourage effect can be a consideration when constructing, formulating, and/or designing a terpene-based, or other, solution for use with the base. The mixing ratios of the described terpene-based solution in relation to the base it is being combined with and adjusting ratios of the various terpenes and terpenoids can create different interactions and benefits than those of the terpenes themselves, as well as the base material they are being mixed with.

The solution 200 can also include a dilutant 290, such as propylene glycol (PG) 291, polyethylene glycol (PEG) 292, vegetable glycerin (VG) 293, and/or other dilutants 290. The dilutant 290 can be a bulker or filler that adds volume to the overall solution 200 and/or dilutes the solution 200 to a desired concentration of components and/or compounds. Additionally, the dilutant(s) 290 can assist with determining the ratio at which the solution 200 and a base are combined to create the product having the required, desired, and/or preferred characteristics and/or properties. A dilutant 290 included in the solution 200 can be selected based on the various base characteristics 210, product characteristics 240, user experience characteristics 270, and/or other characteristics, properties and/or considerations. For example, the dilutant 290 can have a polarity that is incompatible with the polarity of the base the solution 200 is to be combined with. The use of the particular dilutant 290 in the solution 200 can cause the resultant product to separate and/or not stay mixed for the desired and/or an extended period of time. Additionally, the dilutant 290 can have a flavor component and/or profile, which the solution 200 construction and/or formulation can consider when constructing and/or formulating the solution 200.

The various components, constituents and/or compounds that construct and/or formulate the solution 200 can be selected from those that are considered safe for use by a user and/or consumer of the resultant product. Such components, constituents and/or compounds that can include those that are safely ingestible by humans, and at a minimal Generally Regarded As Safe (GRAS) by the Food and Drug Administration (FDA).

The solution 200 can include co-agents, which can include chemicals, substances, or mixtures thereof, in liquid and/or gaseous forms, which can be organic and/or synthetic. The co-agents can assist in the various uses of the product, such as the absorption of the product, formed by at least the combination of the solution 200 and a base, into a living subject, such as a user. For example, the co-agent can function as an emulsifier, a carrier agent, dilutant, inert substance, flavor modifier or substance modifier.

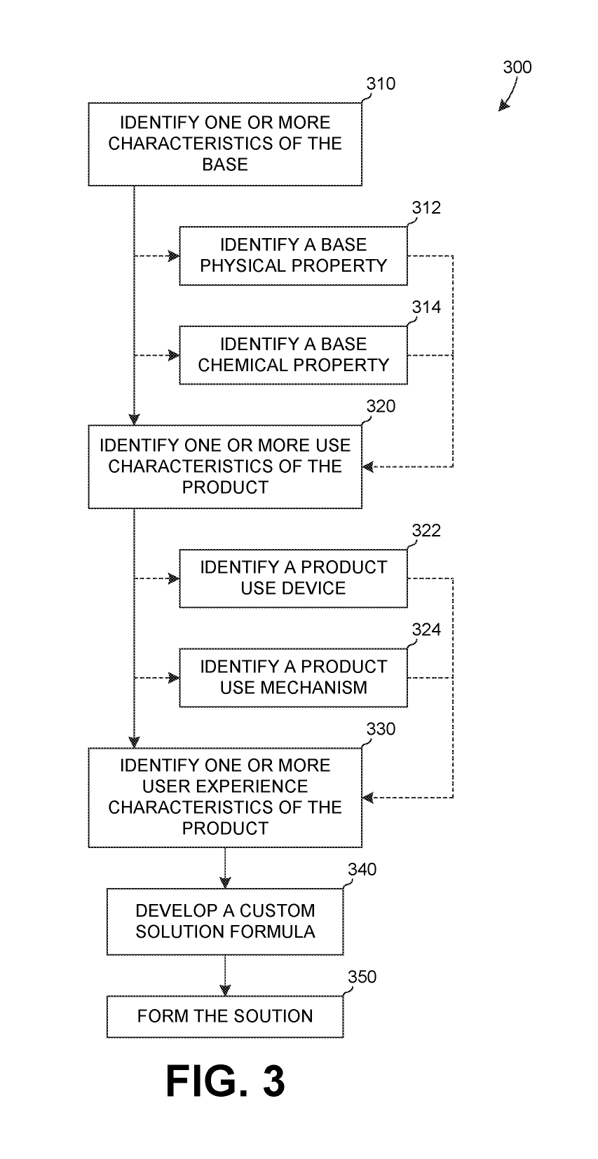

FIG. 3 is an example process 300 for the construction and/or formulation of a solution. The solution can be constructed and/or formulated based on variety of considerations, including required, desired, and/or preferred characteristics and/or properties of a product formed by the combination of the solution and a base, such as a plant extract. The solution can create and/or customize a desired or preferred user experience of the product.

Constructing and/or formulating the solution can include identifying one or more characteristics of the base 310. The base characteristics can include various base properties and/or characteristics. The solution can be constructed and/or formulated to add, enhance, minimize and/or otherwise affect one or more of the identified characteristics of the base. The identified base characteristics 310 can include a physical property 312 and/or a chemical property 314 of the base.

Constructing and/or formulating the solution can also include identifying one or more use characteristics of the product 320. The product is formed by combining the constructed solution and the base, the resultant product can have various characteristics and/or properties. The solution can be constructed and/or formulated based on the various product characteristics 320 which can include the use device 322 and/or a use mechanism 324. The solution, when combined with the base, can create a customized product that is tailored to one or more of the identified product characteristics 320, such as the use device 322 and/or use mechanism 324. A product for use with a particular use device 322 can have various product characteristics 320 associated based on the use device 322 the product is intended to be used with. For another use device 322, the product can have the same or different product characteristics 320. The solution can be constructed and/or formulated to create a product having the required and/or desired product characteristics for use of the product with a particular device 322 and/or a use mechanism 324.

Constructing and/or formulating the solution can further include identifying one or more user experience characteristics of the product 330. The user experience characteristics can include subjective and/or objective components of a user's experience of using the product formed by the combination of at least the base and the solution. The solution can be constructed and/or formulated based on the identified user experience characteristics of the product 330, such as the flavor of the product.

Once the various characteristics of the base 310, the product 320 and/or the user experience 330 have been identified, a custom solution formula can be developed 340. For example, the identified base characteristics 310 can be considered a base profile, what the base is. The identified product characteristics 320 and user experience characteristics 320 can be a product profile, what the product is required and/or desired to have and/or be like. The solution can bridge the gap between the base profile and the product profile, enhancing or minimizing characteristics and/or properties of the base and/or adding additional properties and/or characteristics, such that when the solution and base are combined, the resultant product has the required and/or desired characteristics, i.e. is substantially similar to the product profile.

Once the solution has been formulated 340, the solution can be formed 350, or constructed. The solution can use various terpenes, dilutants and/or other components/compounds to create a solution 350 according to the formula 340. The created solution 350 can then be combined with the base to result in a product that a user can use and/or consume.

### Cannabis Base Example

An example base a solution can be used with can include a cannabis-based base, such as a cannabis extract. The solution can be constructed and/or formulated to mirror and/or compliment the cannabis extract base. The process of creating of such a solution for use with a cannabis extract can include obtaining a premium sample of the cannabis plant that the solution is attempting to recreate. The cannabis plant sample can include a cannabis bud produced by a mature flowering female cannabis plant at the end of the flowering cycle. The cannabis bud can be analyzed, usually by liquid or gas chromatography, to obtain a terpene profile of the cannabis plant. The terpene profile of the submitted plant material can contain a listing of the different terpenes found in the sample and their different ratios. Based on the terpene profile, the top 5-6 terpenes of the cannabis sample can be identified. The relative strength of each terpene of the terpene profile can also be assessed, in regards to aroma and potency of the individual terpenes. For example, one gram of Limonene may smell much stronger than ten grams of pinene, so not only relative concentration of the various terpenes should be considered, but also the relative strength of the various terpenes. The terpene profile may not be enough to accurately recreate the cannabis plant; experience may also be necessary to make terpene concentration adjustments to modify the terpene profile based solution to more closely align with the actual cannabis plant. While the terpene profile can be recreated by simply following what is outlined in the analysis, other considerations, such as other base characteristics of the cannabis extract, use characteristics of the product and/or user experience characteristics, can be considered when creating or designing the solution to be mixed with the cannabis extract base.

Cannabis extracts can include the naturally occurring terpenes found in the cannabis plants. Additional terpenes that are already found in cannabis can blend quickly and easily into the cannabis extract, as the terpenes are not foreign to the initial extract. The addition of terpenes to the cannabis extract base can provide a more favorable and enjoyable experience for a user when using and/or consuming the product formed by the combination of the solution and the cannabis extract base. Terpenes themselves resemble the properties of common alcohols, which have a very low viscosity, this low viscosity requires only a small amount of terpenes to be added to the extract in order to thin the extract to an acceptable level.

Terpenes can also be used to flavor the product of the combination of the solution and cannabis extract base, without the specific intention of diluting the product. The product can contain a ratio of anywhere from 1% to 300% of a terpene-based solution in relation to the plant material it is being combined with. For example, a product can contain one gram of cannabis extract base mixed with one milligram to three grams of a terpene-based solution. As terpenes have a wide variety of strengths of aroma, flavor and taste, a wide range of ratios of terpene, or terpene-based solution, to a cannabis extract base can be used to form the product.

The resultant product of the solution and the cannabis extract base can be intended for use in various delivery and/or use devices and/or mechanisms, such as vaping, smoking and “Dabbing”, a term used to describe the direct incineration and inhalation of plant extracts with a device. The solution can be constructed, formulated, and/or designed to have various flavor profiles as desired. The solution can be used to flavor plant extracts, such as the cannabis extract base, and/or other products as desired. The variety of flavor profiles allow a user to choose or select a solution that is pleasing to their palate and can be an attractive aspect of the various terpene solutions and terpene based products. Flavored solutions can be used to prepare or flavor plant extracts and other products.

The use device and/or mechanism the product of the solution and the cannabis extract base can require and/or desire the product have certain properties and/or characteristics. For example, a vape pen use device and/or a vaporization use mechanism can require or desire the product be a liquid having a low viscosity. The cannabis extract base can be a solid that the solution will need to dissolve and/or otherwise liquefy or suspend in a liquid form. One of the potential issues in liquefying a cannabis extract base for vaporizing, or for use in vaporization devices, is finding a solution which is safe for human consumption, that combines or mixes with the desired cannabis extract base, and will provide an acceptable or desirable user experience. For a solution to provide a product having an acceptable user experience, the solution should mix with the cannabis extract base to form a product that remains blended or suspended over an extended period of time without separation of the various parts or components, the solution should retain a low dilution rate as not to lessen the effects of the cannabis extract base or main plant material ingredient, the product should offer a pleasant taste or not introduce an unpleasant taste, and the solution should be of a food grade material.

The solution can include various dilutants, such as Propylene Glycol (PG), Polyethylene Glycol (PEG) and Vegetable Glycerin (VG). While resulting in a product that produces a user pleasing vapor amount and being acceptable to humans, VG has been found to be non-compatible with cannabis extracts due to conflicting polarity of the cannabis compounds and the VG molecules. The conflicting polarity causes the separation of the VG-extract solution, into VG and extract portions, over a short period of time due to the chemical incompatibility. While both PG and PEG function to dissolve, mix or suspend cannabis extracts, the viscosity of both PG and PEG is so high that a large amount of either is needed to adjust viscosity of the cannabis extract to a desired level. The high ratio of PG or PEG to the cannabis extract alters the taste of the resulting mixture to an unacceptable level and can dilute the extract to an undesirable or ineffective level.

Another dilutant or solvent that has been used in the extract industry are MCTs, or medium chain triglycerides, such as coconut oil. While MCTs are an organic substance that blends with cannabis extracts, does not add an undesirable taste, and stays emulsified or mixed with the extract material, the inhalation of MCTs has been linked to adverse health effects, such as lipid pneumonia. As such, MCTs cannot be considered a suitable ingredient for an inhalation sub stance.

Terpene-based solutions, or other solutions, can be mixed with cannabis, or extracts derived therefrom, to alter the structure of the extract. For example, the terpene-based solution can be used to flavor the cannabis extract, dilute the cannabis extract, adjust the viscosity of the cannabis extract, adjust the solubility of the cannabis extract, increase, or decrease the vapor production of the cannabis extract, or alter the health benefit of the cannabis extract. A cannabis extract can consist of any part of the cannabis plant, such as the trichomes, a whole plant extract, any single compound or group of compounds such as tetrahydrocannabinol (THC), cannabidiol (CBD), cannabinol (CBN), or any individual chemical, compound or substance found in the cannabis plant.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be used for realizing the invention in diverse forms thereof.

1-9. (canceled)

10. A solution for use with a base to form a product including at least the base suspended in the solution, the solution including at least one terpene and formulated based in part on at least one of a base characteristic, including at least one of a physical property and a chemical property, and at least one of a use characteristic, including at least one of a product use device and a product use mechanism.

11. The solution of claim 10, wherein the base physical property includes at least one of a base matter state, a base solubility, a base polarity, a base color, a base viscosity and a base density.

12. The solution of claim 10, wherein the base chemical property includes at least one of a base combustibility, a base toxicity and a base chemical stability.

13. The solution of claim 10, wherein the use device includes at least one of a vape pen, a vaporizer, a heating coil and an aerosolizer.

14. The solution of claim 10, wherein the product use mechanism includes at least one of combustion, vaporization, volatilization and consumption.

15. The solution of claim 10, further including a user experience characteristic, the solution formulated based in part on at least one of the base characteristic, the use characteristic and the user experience characteristic.

16. The solution of claim 15, wherein the user experience characteristic includes at least one of a product taste, a product vapor production, a product odor and a product residuals.

17. The solution of claim 10, wherein the base includes a cannabis extract.

18. The solution of claim 10, wherein the solution includes a dilutant selected from one or more of PG, VG and PEG.

19. (canceled)

20. A solution for use with a base to form a product, wherein the base is analyzed to determine a terpene profile of the base including various terpenes of the base and relative strengths of the various terpenes, the solution formed based at least in part on the determined terpene profile of the base.
